(19) United States
(12) Patent Application Publication
(10) Pub. No.: US 2025/0090721 A1
(43) Pub. Date: Mar. 20, 2025

(54) Charged Citrate-Based Biomaterials

(71) Applicant: Acuitive Technologies, Inc., Allendale, NJ (US)

(72) Inventors: Minh-Tuan Richard Tran, Ho-Ho-Kus, NJ (US); Ruqiang Lu, Ramsey, NJ (US); Angelina Mauriello, Berkeley Heights, NJ (US)

(73) Assignee: Acuitive Technologies, Inc., Allendale, NJ (US)

(21) Appl. No.: 18/891,944

(22) Filed: Sep. 20, 2024

Related U.S. Application Data
(60) Provisional application No. 63/539,378, filed on Sep. 20, 2023.

(51) Int. Cl. A61L 27/18; A61L 27/54; A61L 27/56; A61L 27/58

(52) U.S. Cl. CPC A61L 27/18; A61L 27/54; A61L 27/56; A61L 27/58; A61L 2300/252; A61L 2300/414; A61L 2300/602; A61L 2430/02

(57) ABSTRACT

Charged citrate-based compositions are provided for use in regenerative applications. The disclosed compositions generally include (i) a citrate component, (ii) a diol, and/or (iii) a charged moiety. The compositions may be used to fabricate scaffolds. The citrate component may take the form of citric acid, citrate, or ester of citric acid, the polyol may take the form of a diol, such as butanediol, hexanediol, octanediol, glycerol or xylitol, and the charged moiety is an anionic or cationic moiety. The anionic moiety may take the form of sulfate, sulfonate, phosphate, phosphonate, sulfamate, or carboxylic acid, and the cationic moiety may take the form of quaternary ammonium, pyridinium, piperidinium, pyrrolidinium, imidazolium, or phosphonium. The anionic or cationic moiety can be presented in the polyol. The composition may include a particulate inorganic material, such as hydroxyapatite, tricalcium phosphate, biphasic calcium phosphate, or bioglass.

### CROSS-REFERENCE TO RELATED APPLICATION

The present application claims priority benefit to a U.S. provisional application entitled “Charged Citrate-Based Biomaterials” that was filed on Sep. 20, 2023, and assigned Ser. No. 63/539,378. The entire content of the foregoing provisional application is incorporated herein by reference.

### BACKGROUND

### Technical Field

The present disclosure is directed to charged citrate-based compositions to control the binding and release of therapeutic molecules in regenerative engineering applications.

### Background Art

Despite bone's capacity to regenerate after injury, delayed healing or nonunion, is a significant clinical challenge in orthopedics (Chen C. H., Hsu E. L., Stupp S. I. Supramolecular self-assembling peptides to deliver bone morphogenetic proteins for skeletal regeneration. Bone. Volume 141, December 2020, 115565). Out of the 8 million fracture repair cases which occur annually in the U.S., over 10% resulted in nonunion and significantly higher rates have been reported for smokers, diabetics, and osteoporotic patients (Buza J. A. 3rd; Einhorn T. Bone healing in 2016. Clinical Cases Mineral and Bone Metabolism 2016, 13(2), 101-105).

Autogenous bone grafting from the iliac bone has been considered the clinical gold standard treatment for bone formation in the repair of large bone defects and fusion applications (Dohzono S., Imai Y., Nakamura H., Wakitani S., Takaoka K. Successful spinal fusion by E. coli-derived BMP-2-adsorbed porous beta-TCP granules: a pilot study. Clinical Orthopaedics and Related Research. December 2009, 467(12), 3206-3212). However, donor site deformity, pain, infection, and supply limitations are major disadvantages associated with the harvesting of iliac bone graft (Arrington E. D., Smith W. J., Chambers H. G., Bucknell A. L., Davino N. A. Complications of iliac crest bone graft harvesting. Clinical Orthopaedics and Related Research. 1996, 329, 300-309). These limitations in combination with developments in bone biology have driven researchers towards alternative approaches using bioactive molecules and materials.

For example, bone morphogenic protein (BMP) is a naturally occurring bioactive molecule capable of ectopic bone formation (Urist M. R. Bone—formation by Autoinduction. Science. 1965, 150(3698), 893-899). While healing rates as high as 92% have been reported in nonunion patients (Zhou Y. Q., Tu H. L., Duan Y. J., Chen X. Comparison of bone morphogenetic protein and autologous grafting in the treatment of limb long bone nonunion: a systematic review and meta-analysis. Journey of Orthopedic Surgery and Research. 2020, 15(1), 288), adverse effects such as inflammatory reactions, soft tissue hematomas, bone cysts, ectopic bone formation, cancer, retrograde ejaculation, infections, and radiculopathy have been connected to BMP treatment (El Bialy I., Jiskoot W., Reza Nejadnik M. Formulation, Delivery and Stability of Bone Morphogenetic Proteins for Effective Bone Regeneration. Pharmaceutical Research. 2017, 34(6), 1152-1170). It has been widely reported that the main reason behind these adverse effects is the high dose of administered BMP (Mroz T. E., Wang J. C., Hashimoto R., Norvell D. C. Complications related to Osteobiologics use in spine surgery a systematic review. Spine. 2010, 35(9), S86-S104.). For example, Medtronic's INFUSE bone graft kit utilizes 1.5 mg/mL of BMP-2, which exceeds one million times the physiological concentrations during normal bone repair conditions (Gamradt S. C., Lieberman J. R. Genetic modification of stem cells to enhance bone repair. Annals Biomedical Engineering. 2004, 32(1), 136-147).

It has been shown that the efficacy and some of the reported side effects of BMPs can be controlled by improving their delivery system. Current BMP delivery systems are based on collagen due to its biocompatibility and ability to be fabricated as powders, films, gels, fibers, and absorbable sponges (Lee S. H., Shin H. Matrices and scaffolds for delivery of bioactive molecules in bone and cartilage tissue engineering. Advanced Drug Delivery Review. 2007, 59(4-5), 339-359.). However, it has been shown in vivo that only 5% of BMP remains within collagen sponges after 2 weeks due to an initial burst release (Geiger M., Li R. H., Friess W. Collagen sponges for bone regeneration with rhBMP-2. Advanced Drug Delivery Review. 2003, 55(12), 1613-1629). To address this limitation, significant research activity has been focused on the design of biomaterial delivery systems with improved protein binding capabilities to reduce the dosage of BMP-2. Many studies have attempted to reduce the dosage and decrease the release rate of BMP-2 by binding heparin to their carriers. Heparin is a highly sulfated glycosaminoglycan that is negatively charged, which causes electrostatic interactions with growth factors like BMP-2, forming a polyelectrolyte complex (Terauchi M., Tamura A., Tonegawa A., Yamaguchi S., Yoda T., Yui N. Polyelectrolyte complexes between polycarboxylates and BMP-2 for enhancing osteogenic differentiation: effect of chemical structure of polycarboxylates. Polymers. 2019, 11(8), 1327-1327). However, the main disadvantage to using heparin to attract growth factors is its anticoagulant effect, which could increase the risk of complications during surgical operation and healing (Terauchi et al.).

Therefore, a need exists for biocompatible compositions with improved protein binding and controlled protein releasing capabilities for regenerative engineering applications, e.g., replacements for injured tissues.

### SUMMARY

According to the present disclosure, highly advantageous charged citrate-based compositions are fabricated and utilized to control the binding and release of therapeutic molecules.

In exemplary embodiments, the disclosed composition comprises (i) a citrate component, (ii) a polyol, and/or (iii) a charged moiety. The citrate component may comprise one or more of citric acid, citrate, or ester of citric acid. The polyol may comprise a diol, e.g., one or more of butanediol, hexanediol, octanediol, or polyethylene glycol. Other exemplary polyols contemplated according to the present disclosure include one or more of glycerol, beta-glycerol phosphate, or xylitol.

The anionic moiety may comprise one or more of sulfate, sulfonate, phosphate, phosphonate, sulfamate, or carboxylic acid. In some exemplary embodiments, the anionic moiety can be present in the polyol. In certain exemplary embodiments, the sulfate, sulfonate, phosphate and phosphonate containing moiety may further comprise one or more of hydroxyl, amine, or carboxylic acid. In other exemplary embodiments, the sulfate and sulfonate containing moiety include one or more of taurine, isethionic acid, or sodium 2-carboxyethane-1-sulfonate. In a specific example, the phosphate and phosphonate containing moiety may comprise 3-(phosphonooxy) propanoic acid or 2-hydroxyethyl phosphate. In exemplary embodiments, the cationic moiety may comprise one or more of quaternary ammonium, imidazolium, pyridinium, piperidinium, pyrrolidinium or phosphonium. In certain exemplary embodiments, the cationic moiety include (2-aminoethyl)trimethylammonium chloride and choline chloride. In other exemplary embodiments, the charged moiety may further comprise one or more of alkene, alkyne, halogen, borate, or azide.

In some exemplary embodiments, the charged moiety may be conjugated via amidation reaction, esterification, and/or substitution reaction. In a more specific example, the amidation or esterification reaction may be assisted. In some embodiments, the amidation reaction is assisted by carbodiimide reagents, uranium or aminium activating groups, N′,N′-dicyclohexyl carbodiimide (DCC), dimethylaminopyridine (DMAP), succinimide reagents, or N,N′-carbonyldiimidazole (CDI). In exemplary embodiments, the amidation reaction is assisted by 1-ethyl-3-(-3-dimethylaminopropyl) carbodiimide hydrocholoride (EDC) and N-hydroxysuccinimide.

In some exemplary embodiments, the composition binds proteins or protein mimicking peptides. In exemplary embodiments, the protein or protein mimicking peptide is one or more of bone morphogenic protein (BMP), basic fibroblast growth factor (bFGF), transforming growth factor beta (TGF-β 1), vascular endothelial growth factor (VEGF), platelet derived growth factor (PDGF), stromal cell-derived factor 1 (SDF-1), monocyte chemoattractant protein-1 (MCP-1), interleukins, neuronal growth factor (NGF), brain-derived neurotrophic factor (BDNF), tumor necrosis factor a (TNF-a), heparin-binding epidermal growth factor (HB/EGF), sonic hedgehog (SHH), and/or epidermal growth factor (EGF). In a more specific example, the BMP may comprise BMP-2, BMP-4, or BMP-7. In other embodiments, the composition can bind other charged molecules such as antibiotics, chemokines, and steroids. In some exemplary embodiments, the composition binds proteins via a covalent attachment. In some embodiments, the protein or protein mimicking peptide is attached via a biodegradable attachment. In some exemplary embodiments, the protein or protein mimicking peptide is attached via an amide or ester attachment. In some exemplary embodiments, the covalently attached protein or protein mimicking peptide is BMP-2.

In exemplary embodiments, the composition may further comprise a particulate inorganic material. The disclosed particulate inorganic material may comprise one or more of hydroxyapatite, tricalcium phosphate, biphasic calcium phosphate, or bioactive glass. In some embodiments, the particulate inorganic material is present in an amount between 10 and 60 wt.-% of the composition. In certain embodiments, the particulate inorganic material is micro or nano-sized. In some exemplary embodiments, the particulate inorganic material is rod-shaped. In other exemplary embodiments, the particulate inorganic material is fiber-shaped.

In some exemplary embodiments, a scaffold can be formed at least in part from the disclosed composition, wherein the scaffold is biodegradable. In some embodiments, the scaffold is 50-95% porous. In exemplary embodiments, the scaffold contains a gradient porous structure.

Additional features, functions, and benefits of the disclosed citrate-based compositions will be apparent from the following description of exemplary embodiments.

### BRIEF DESCRIPTION OF THE FIGURES

To assist those of skill in the art in making and using the disclosed compositions, systems and methods, reference is made to the accompanying figures, wherein:

FIG. 1 is a schematic structure of POXC-IA;

FIG. 2 is 1H NMR spectra of POXC-IA and POXC-control showing addition of 1A;

FIG. 3 is a schematic depiction of surface conjugation of sulfate and/or sulfonate to citrate-based biomaterial scaffold using (A) free carboxylic acid, and (B) hydroxyl group;

FIG. 4A depicts poly (octamethylene-xylitol citrate) (POXC) scaffolds surface modified to present sulfate and/or sulfonate groups.

FIG. 4B is high-resolution X-ray photoelectron spectroscopy (XPS) spectra of S2p;

FIG. 4C shows sulfate/sulfonate content obtained from the integration of high-resolution XPS spectra of S2p and C1s (defined as the ratio of S atom and C atom contents);

FIG. 4D shows surface zeta-potentials of INFUSE collagen, POXC, POXC-IA-Ta and POXC-IA-SO3Na scaffolds under different pHs;

FIG. 5 shows cumulative rhBMP-2 release from INFUSE collagen scaffolds and sulfate/sulfonate modified POXC scaffolds;

FIG. 6 shows a first week of cumulative rhBMP-2 release from INFUSE collagen scaffolds and sulfate/sulfonate modified POXC scaffolds; and

FIG. 7 shows cumulative rhPDGF-BB release from sulfate/sulfonate modified POXC scaffolds.

### DESCRIPTION OF EXEMPLARY EMBODIMENTS

As noted above, the present disclosure is related to highly advantageous charged citrate-based compositions for controlled binding and releasing therapeutic molecules. In certain embodiments, the present disclosure provides a composition comprising (i) a citrate component, (ii) a polyol, and/or (iii) a charged moiety.

Citric acid is an inexpensive, nontoxic, and naturally occurring metabolic molecule that participates in bone anatomy and physiology by regulating the growth of apatite nanocrystals and cellular energy production. Furthermore, citrate-based biomaterials present valuable chemical functional groups for bulk chemistry modification and surface conjugation chemistry. In addition to the citrate chemistry, citrate-based biomaterials provide unique elastomeric properties that can be conducive for native tissue development during regeneration. As used herein, the terms “citrate-based compositions” and “citric acid-based biomaterials” are interchangeable and refer to a polymeric compound produced by reacting citric acid with a polyol monomer to generate a polymer having a backbone that comprises hydrolysable ester bond.

Citrate-based biomaterials are rich in carboxyl and hydroxyl bulk chemistry, which can be modified with anionic moiety. In exemplary embodiments, the anionic moiety may comprise one or more of sulfate, sulfonate, phosphate, phosphonate, sulfamate, or carboxylic acid. For example, sulfate and sulfonate containing moieties, including but not limited to taurine or its salt form and isethionic acid or its salt form, can react with the free carboxylic acid groups of citric acid/citrate through esterification or amidation reaction.

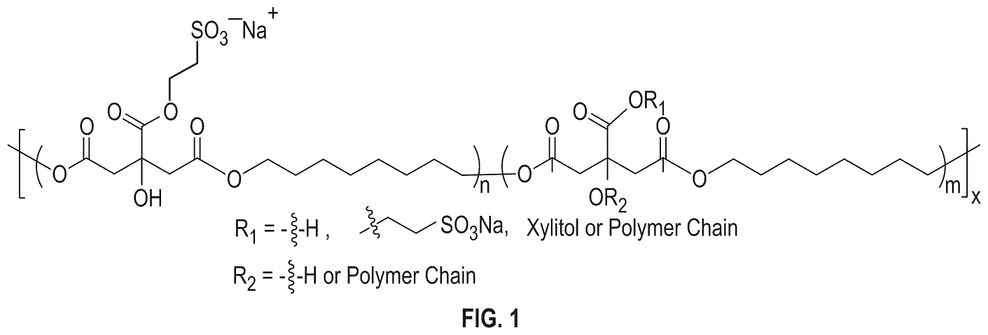

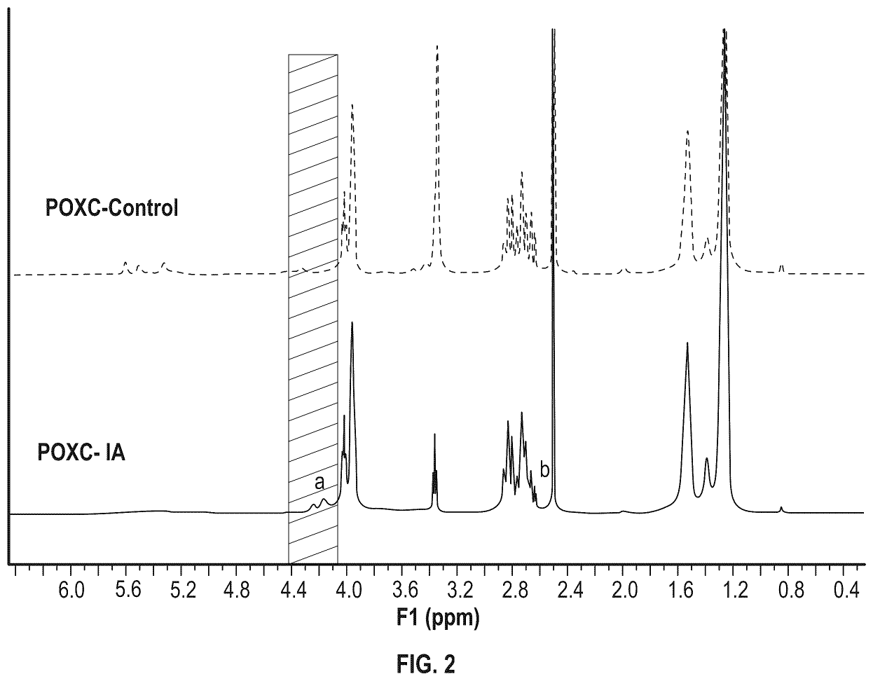

As an example, the addition of isethionic acid sodium salt (IA) to the mixture of citric acid, xylitol, and 1,8-octandiol leads to poly (octamethylene-xylitol citrate) (POXC) with isethionic acid sodium salt to form POXC-IA (FIG. 1). Proton nuclear magnetic resonance (¹H NMR) characterization of the pre-polymer shows successful introduction of the sulfonate moiety in the citrate-based polymer, i.e. POXC-IA (FIG. 2). As shown in FIG. 2, the characteristic peaks from isethionic acid moiety are at 4.22-4.16 ppm and 2.70-2.62 ppm.

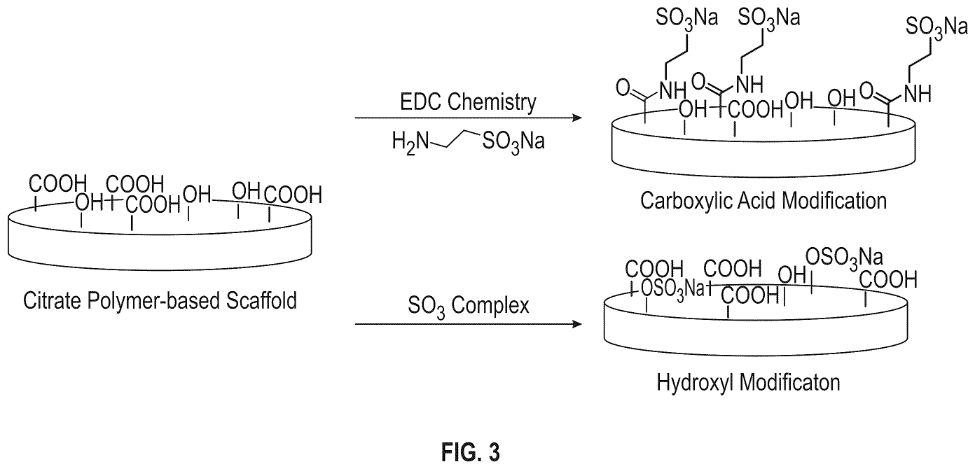

In other exemplary embodiments, sulfates and/or sulfonates can be conjugated to the surface of citrate-based biomaterials using free carboxylic acid and/or hydroxyl group (FIG. 3).

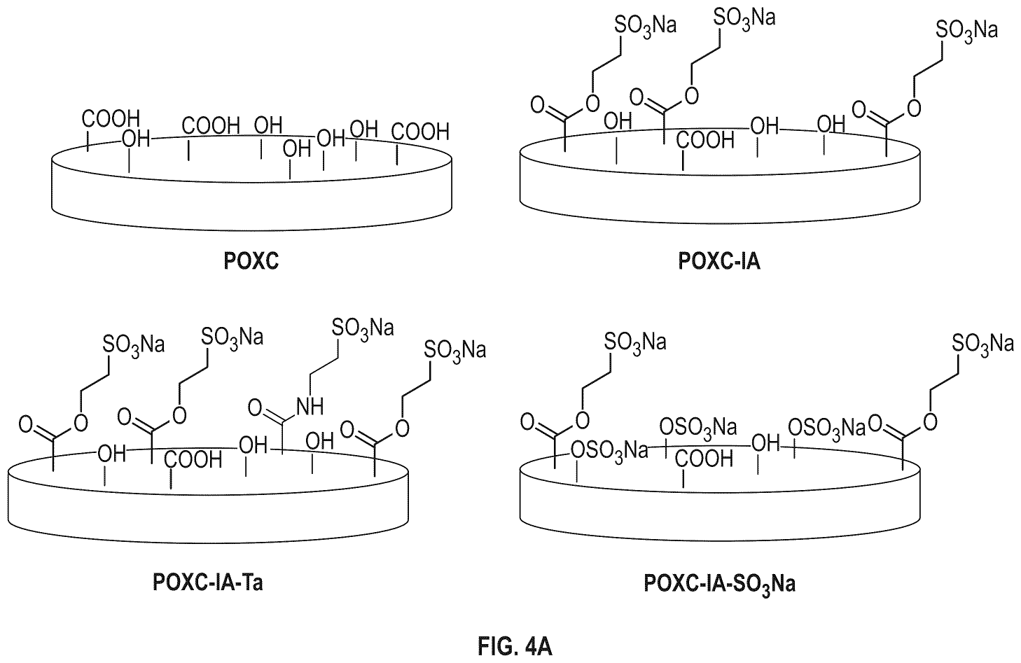

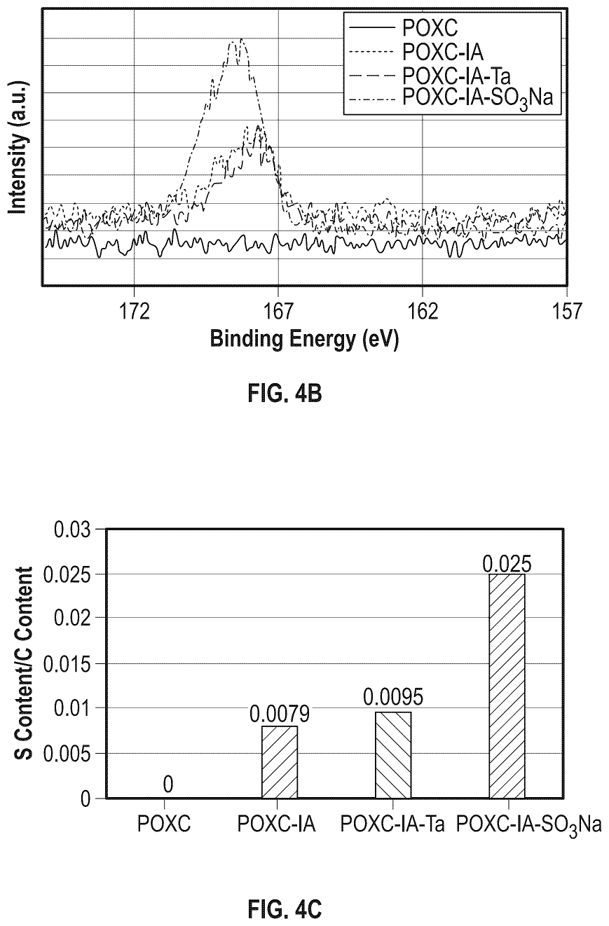

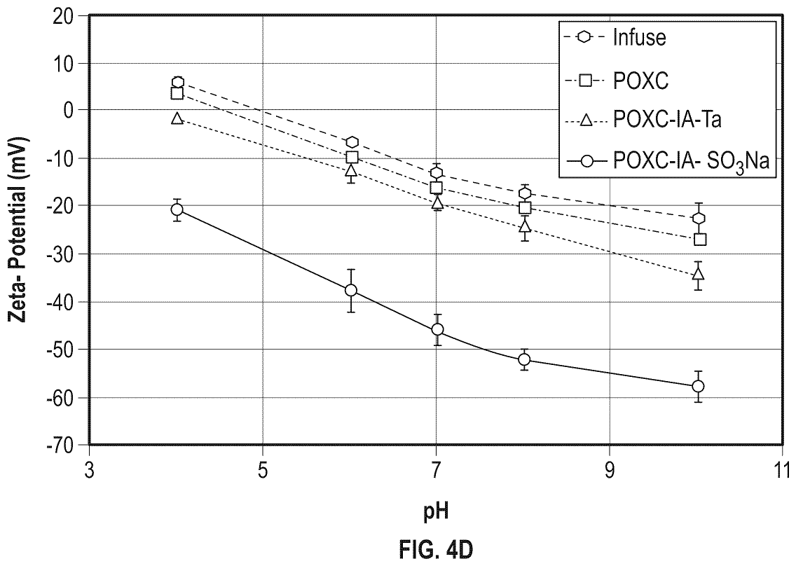

FIG. 4 shows exemplary embodiments of modified citrate-based polymer scaffolds with sulfate and/or sulfonate containing moiety. To make porous polymer scaffolds, POXC or POXC-IA pre-polymer was mixed with sodium chloride and then cured at 80° C. for 7 days. Following polymer crosslinking, the salt was leached out with deionized water. The obtained POXC-IA scaffolds may be soaked in a 1-ethyl-3-(3-dimethylaminopropyl)carbodiimide (EDC)/N-hydroxysuccinimide (NHS)/Taurine solution and shaken at room temperature for 24 hours to form scaffolds with both isethionic acid (IA) and taurine sodium salts (Ta), as shown in FIG. 4A as POXC-IA-Ta. Successful surface conjugation was confirmed by X-ray photoelectron spectroscopy (XPS), which showed an increased sulfur peak intensity (FIGS. 4B and 4C). As shown in FIG. 4B, the S2p peak at about 168 eV corresponds to —SO₃⁻. The sulfonate and/or sulfate content was defined as the ratio of S atom and C atom content (S/C) obtained from the integration of high-resolution XPS spectra of S2p and C1s. As shown in FIG. 4C, the S/C ratio increases from 0 in POXC scaffold to 0.0079 in POXC-IA and to 0.0095 in POXC-IA-Ta.

As another example, surface modification of POXC-IA scaffolds can be achieved by soaking in a sulfur trioxide trimethylamine complex in DMF at 35° C. for 24 hours. The substitution reaction on the pendant hydroxyl groups leads to scaffolds with both isethionic acid and sulfate groups, as shown in FIG. 4A, POXC-IA-SO₃Na. As shown in FIGS. 4B and 4C, XPS characterization confirms successful surface modification with a significant increase in the sulfur content. As shown in FIG. 4C, the S/C ratio in POXC-IA-SO₃Na increases to 0.025. Of note, the C atom content for POXC-IA-Ta was corrected via the subtraction of the C atom from taurine.

In exemplary embodiments, the sulfate/sulfonate contents may be further tuned by the amount of sulfation reagents and/or reaction temperature. The content of sulfate/sulfonate effectively modulates the surface charges of those citrate-polymers-based scaffolds. As shown in FIG. 4D, the surface zeta-potential becomes more negative with the increasing sulfate/sulfonate content under pH 4 to 10. Note that the surface zeta-potentials of POXC-based scaffolds are more negative than the commercially available INFUSE collagen sponge.

To show the ability of sulfate and/or sulfonate modified citrate-based materials to bind and release proteins, recombinant human BMP-2 (rhBMP-2) and recombinant human platelet derived growth factor (rhPDGF-BB) were used as model proteins.

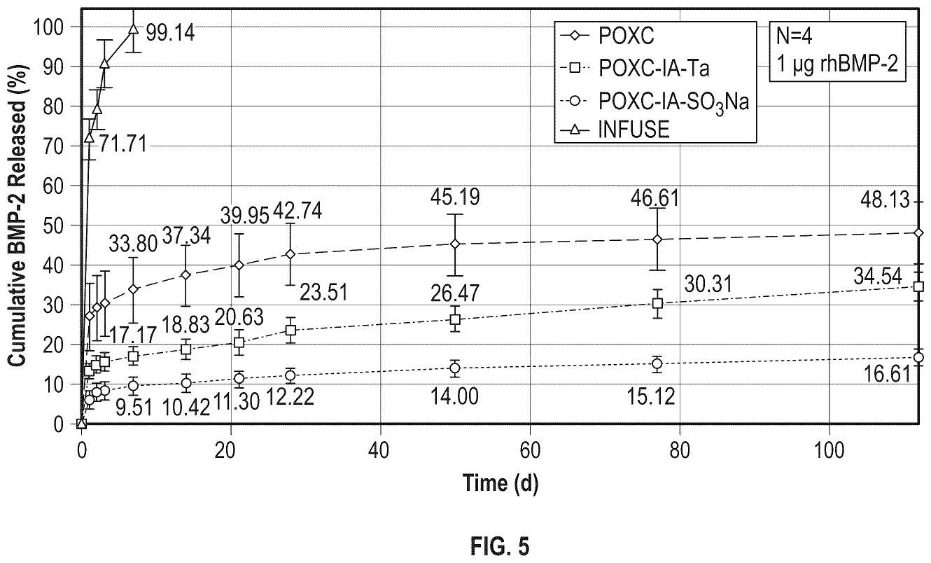

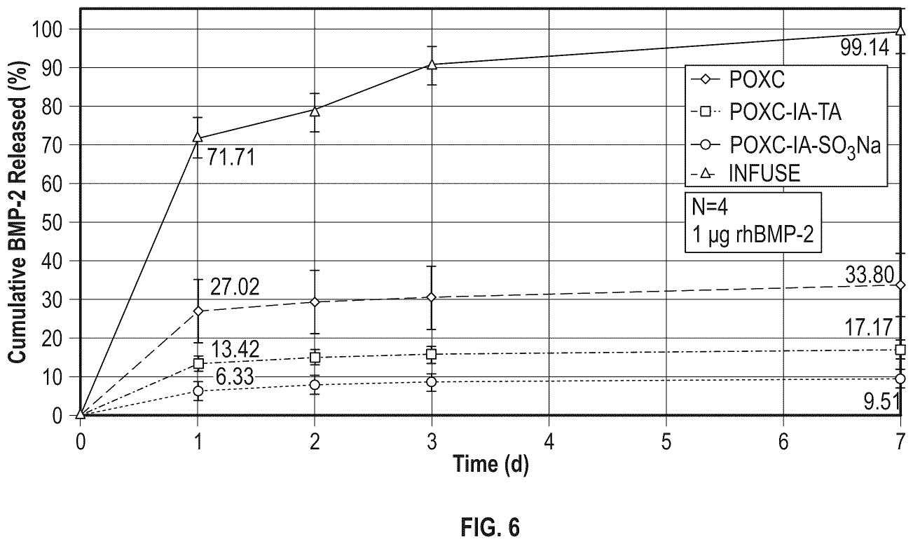

Briefly, 1 μg of rhBMP-2 was loaded onto INFUSE collagen scaffolds, POXC scaffolds, and sulfation/sulfonation POXC scaffolds modified with different sulfation/sulfonation degrees. Cumulative rhBMP-2 release was measured on day 1, 2, 3, 7, 14, 21, 28, 50, 77 and 112 using a BMP-2 ELISA Kit (R&D Systems, Minneapolis, MN). As shown in FIGS. 5 and 6, rhBMP-2 release from INFUSE collagen scaffolds resulted in an immediate burst release of 71.17±5.16% after 24 hours. After 7 days, 99.14±5.72% is released from the INFUSE collagen scaffolds. In contrast, rhBMP-2 release from POXC scaffold and modified POXC scaffolds was slower and controlled with the degree of sulfation/sulfonation. Those results are consistent with the surface zeta-potentials, confirming the surface charge plays a key role in controlling rhBMP-2 binding and release.

For example, as shown in FIG. 6, after 24 hours, rhBMP-2 release was 27.02±8.26%, 13.42±2.01%, and 6.33±2.29% for POXC, POXC-IA-Ta (S/C=0.0095) and POXC-IA-SO₃Na (S/C=0.025) scaffolds, respectively. A slow and steady rhBMP-2 release continued after 112 days, with 48.13±7.71% of rhBMP-2 released from the POXC, 34.54±3.43% of rhBMP-2 released from the POXC-IA-Ta scaffolds, and 16.61±2.12% released from the POXC-IA-SO3Na scaffolds, respectively.

Using the same method of binding and release of rhBMP-2, rhPDGF-BB was loaded onto the sulfation/sulfonation POXC scaffolds modified with different sulfation/sulfonation degree. The data in FIG. 7 shows the cumulative release of rhPDGF-BB over 1, 3, 7, 14, 28, and 56 days using a rhPDGF-BB ELISA kit (R&D Systems, Minneapolis, MN). After 24 hours, the rhPDGF-BB release was 14.54±4.54%, 2.14±0.37%, and 2.97±1.91% for POXC, POXC-IA-Ta (S/C=0.0095) and POXC-IA-SO₃Na (S/C=0.025) scaffolds, respectively. The release of rhPDGF-BB was slower than the release of rhBMP-2, possibly because rhPDGF-BB has a higher isoelectric point and lower molecular weight, causing it to be more positively charged (Niu, W.; Lim, T. C.; Alshihri, A.; Rajappa, R.; Wang, L.; Kurisawa, M.; Spector, M. Platelet-Derived Growth Factor Stimulated Migration of Bone Marrow Mesenchymal Stem Cells into an Injectable Gelatin-Hydroxyphenyl Propionic Acid Matrix. Biomedicines 2021, 9, 203). After 56 days, the rhPDGF-BB release was 19.97±4.44%, 4.92±0.55%, and 4.86±1.91% for POXC, POXC-IA-Ta (S/C=0.0095) and POXC-IA-SO₃Na (S/C=0.025) scaffolds, respectively.

It has been proven that using certain combinations of growth factors may help bone healing (Almubarak, S.; Nethercott, H.; Freeberg, M.; Beaudon, C.; Jha, A.; Jackson, W.; Marcucio, R.; Miclau, T.; Healy, K.; Bahney, C. Tissue engineering strategies for promoting vascularized bone regeneration. Bone 2016, 83, 197-209 and Kim, Sung Eun, Young-Pil Yun, Jae Yong Lee, June-Sung Shim, Kyeongsoon Park, and Jung-Bo Huh. Co-delivery of platelet-derived growth factor (PDGF-BB) and bone morphogenic protein (BMP-2) coated onto heparinized titanium for improving osteoblast function and osteointegration. J. Tissue Eng. Regen. Med. 2013, E219-E228). The presented materials are also capable of presenting two or more growth factors to achieve simultaneous or sequential release. For example, to achieve the sequential presentation of PDGF and BMP-2, PDGF can be electrostatically bound to the scaffold and released, while BMP-2 can be covalently coupled to the materials scaffold via EDC chemistry. The covalently bound BMP-2 is expected to release more slowly than the electrostatically bound PDGF. To avoid the possible alteration in bioactivity of BMP-2 after covalent coupling, the BMP-2 mimicking peptide can be used instead. The controlled release of growth factors could therefore be achieved by the above-mentioned methods.

Based on these findings, one of ordinary skill would expect similar outcomes using moieties containing other anions including, but not limited to, phosphates, phosphonates, sulfamates, and carboxylates. This is due to the ability of these anions to increase the surface charges in a similar fashion to the sulfates and sulfonates presented in this disclosure. Finally, one would also expect other heparin-binding growth factors such as bFGF, TGF-01, VEGF, and EGF to be sequestered by the chemistries presented above.

In addition to anionic charges, citrate-based biomaterials can be modified to incorporate cationic charges using the rich carboxyl and hydroxyl bulk chemistry. In exemplary embodiments, the cationic moiety may comprise quaternary ammonium, imidazolium, pyridinium, piperidinium, pyrrolidinium or phosphonium. Examples of cationic moieties include but are not limited to (2-aminoethyl)trimethylammonium chloride and choline chloride. Due to the versatile and tunable charge of this material, negatively charged small molecules could be attached. Some exemplary embodiments of this complex include the attachment of negatively charged antibiotics such as penicillin, hormones, steroids like corticosteroids, glycosaminoglycans like hyaluronic acid, and other small molecule drugs.

Although the present disclosure has been described with reference to exemplary embodiments and implementations, the present disclosure is not limited by or to such exemplary embodiments/implementations.

1. A composition comprising:
(i) a citrate component,
(ii) a polyol, and/or
(iii) a charged moiety.

2. The composition of claim 1, wherein the citrate component comprises one or more of citric acid, citrate, or ester of citric acid.

3. The composition of claim 1, wherein the polyol comprises a diol.

4. The composition of claim 3, wherein the diol comprises one or more of butanediol, hexanediol, octanediol, glycerol, xylitol or polyethylene glycerol.

5. The composition of claim 1, wherein the charged moiety comprises an anionic moiety that comprises one or more of sulfate, sulfonate, phosphate, phosphonate, sulfamate, or carboxylic acid.

6. The composition of claim 1, wherein the charged moiety comprises a cationic moiety that comprises one or more of quaternary ammonium, pyridinium, piperidinium, pyrrolidinium, imidazolium, or phosphonium.

7. The composition of claim 1, wherein the charged moiety further comprises one or more of hydroxyl, amine, or carboxylic acid.

8. The composition of claim 1, wherein the charged moiety is conjugated via amidation reaction, esterification, or substitution reaction.

9. The composition of claim 1, wherein the charged moiety is presented in the polyol.

10. The composition of claim 9, wherein the charged moiety is an anionic moiety, and wherein the polyol presented with the anionic moiety includes beta-glycerol phosphate, sulfated xylitol, and sulfated glycerol.

11. The composition of claim 1, wherein a peptide is conjugated to a surface of the composition.

12. The composition of claim 11, wherein the peptide is a protein binding peptide.

13. The composition of claim 11, wherein the peptide is a protein mimicking peptide.

14. The composition of claim 1, further comprising a particulate inorganic material.

15. The composition of claim 14, wherein the particulate inorganic material comprises one or more of hydroxyapatite, tricalcium phosphate, biphasic calcium phosphate, or bioglass.

16. The composition of claim 14, wherein the particulate inorganic material is present in an amount between 10 and 60 wt.-% of the composition.

17. The composition of claim 14, wherein the particulate inorganic material is micro or nano-sized.

18. The composition of claim 14, wherein the particulate inorganic material is rod-shaped or fiber-shaped.

19. A scaffold formed at least in part from the composition of claim 1, wherein the scaffold is biodegradable.

20. The scaffold of claim 19, wherein the scaffold is 50-95% porous.

21. The scaffold of claim 19, wherein the scaffold contains a gradient porous structure.

22. The composition of claim 1, wherein the composition is adapted to bind a therapeutic molecule through a non-covalent interaction.

23. The composition of claim 22, wherein the therapeutic molecule is one or more of a bone morphogenic protein (BMP), a basic fibroblast growth factor (bFGF), a transforming growth factor beta (TGF-β 1), a vascular endothelial growth factor (VEGF), a platelet derived growth factor (PDGF), an epidermal growth factor (EGF), a stromal cell-derived factor 1 (SDF-1), a monocyte chemoattractant protein-1 (MCP-1), an interleukin, a neuronal growth factor (NGF), a brain-derived neurotrophic factor (BDNF), a tumor necrosis factor a (TNF-α), an heparin-binding epidermal growth factor (HB/EGF), a sonic hedgehog (SHH), a charged antibiotic, an hormone, a steroid, a glycosaminoglycan, or other small molecule drug.

24. The composition of claim 23, wherein the BMP comprises one or more of BMP-2, BMP-4, or BMP-7.
